(19) United States
(12) Patent Application Publication
(10) Pub. No.: US 2007/0122366 A1
(43) Pub. Date: May 31, 2007

(54) Preparations for the care nails that contain Terbinafine hydrochloride

(76) Inventor: Fernando Ahumada-Ayala, Mexico (MX)

Correspondence Address: Charles N.J. Ruggiero;Ohlandt, Greeley, Ruggiero & Perle, L.L.P., 10th Floor, One Landmark Square, Stamford, CT, 06901-2682, US

(21) Appl. No.: 11/397,245

(22) Filed: Apr. 4, 2006

(30) Foreign Application Priority Data
Nov. 30, 2005 (MX) PA/A/2005/012961

(51) Int. Cl. A61K 8/40; A61K 31/137

(52) U.S. Cl. 424/61; 514/649

(57) ABSTRACT

There are provided topical preparations for the care of fingernails and toenails. The preparation is in the form of a varnish solution that contains Terbinafine Hydrochloride as an active ingredient and a vehicle formulated with all or some of the following components: ethyl alcohol, poly (methyl vinyl ether alt maleic acid monobutyl ester) in 50% ethanol solution, and Butylated Hydroxytoluene (BHT). The topical preparation can be applied in such a way as to be spread freely on the fine tissue of the nail. The preparation is comfortable when applied to the patient, and can be applied infrequently, depending on the degree of severity of the infection, and also shows good tolerability.

### CROSS REFERENCE TO RELATED PATENT APPLICATIONS

The instant patent application claims priority to Mexican Patent Application No. PA/a/2005/012961, filed on Nov. 30, 2005, which is herein incorporated by reference in its entirety.

### FIELD OF INVENTION

The foregoing invention regards a topical preparation for the care of nails in the form of a varnish solution with fungicidal activity containing Terbinafine Hydrochloride (I) as an active ingredient along with a polymeric film formed by poly (methyl vinyl ether alt maleic acid monobutyl ester) in 50% ethyl alcohol solution, preservatives, and antioxidants

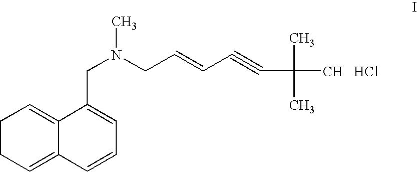

The active compound of the formula may be in the form of salt on an addition of acid, or in free form. The most convenient form is that of a hydrochloride. Its generic name is Terbinafine and is commercially available under the registered brand LAMISIL.

### BACKGROUND OF INVENTION

Antifungal agents are prescribed for mycotical infections in fingernails and toenails caused by dermatophytes like Trichophytons like T rubrum, T. mentagrophytes, T. verrucosum, T. violaceum; Microsporum canis; and Epidermophyton floccosum.

Terbinafine is an allylamine synthetic derivative with broad spectrum antifungal activity. It is a fungicide for dimorphous filamentous fungi, demataceas and some types of yeast. Its activity against yeast is fungicidal or fungistatic, depending on the species.

Terbinafine is an allylamine with fungicidal activity, first approved for the treatment of onychomycosis in England in the early 1990's and in the United States in 1996. Terbinafine is frequently used for oral prescriptions as an antifungal agent in onychomycosis. Its efficacy and safety in the treatment of onychomycosis of the nails in adults is supported in the following studies.

One of them shows that 18 randomized controlled trials prove that it is highly effective with an average mycological cure of 76%. Terbinafine activity is greater than itraconazole and fluconazole in comparative studies for the treatment of toenail onychomycosis. Recent studies have revealed that Terbinafine is more cost-effective than griseofulvin, fluconazole or itraconazole. Terbinafine has been used effectively and safely for the treatment of onychomycosis in special patient populations such as children, the elderly, immunocompromised patients, diabetics, and those with Down syndrome. Terbinafine should be considered as the main active ingredient for the treatment of onychomycosis based in its effectiveness and natural broad spectrum fungicidal activity. (Gupta A K, Ryder J E, Lynch L E, Tavakkol A, J. Drugs Dermatol., May-June 2005; 4(3): 302-8).

Tinea of the skin and nails is a common problem in indigenous communities in the superior corner or Australia, where Terbinafine was found to be a well-tolerated and effective treatment in oral and topical prescriptions. (Koh K J, Parker C J, Ellis D H, Australas. J. Dermatol., November 2003; 44(4): 243-9).

Terbinafine has demonstrated an excellent fungicidal activity against dermatophytes, and a variable activity against yeast. After the oral administration of Terbinafine, it is quickly absorbed and extensively distributed to the fine tissue of the body including the nail matrix. The concentration of Terbinafine in nails is detected within the first week after therapy started. It persists for at least 30 weeks after the treatment has finished. (Darkes M J, Scott L J, Goa K I, Am. J. Clin. Dermatol., 2003; 4(1):39 - 65).

Terbinafine impedes the synthesis of ergosterol through the specific and selective inhibition of fungal squalene epoxidase. This results in a deficit of ergosterol and an accumulation of squalene, which could result in mycotic cellular death. Terbinafine does not influence the metabolism of hormones or other drugs because the enzyme squalene epoxidase is not linked to the cytochrome P450 system. Oral and topical therapy, which is documented in several clinical reports, has demonstrated efficacy and safety in onychomycosis and dermatomycosis, which are susceptible to topical treatments, including chronic or recurring tinea pedis, interdigital mycosis, inguinal tinea, pityriasis versicolor, and cutaneous candidiasis.

Besides its efficacy against dermatophytes after oral and topical administration, it has also been found highly effective against the treatment of onychomycosis, because it has a strong fungicidal activity and a high affinity to nail keratin, where it gets enriched.

The systemic treatment of onychomycosis has some disadvantages, for example, the exposure of the drug's substance to the organism and the need to handle high dosages. Thus, the possibility of having a topical treatment is very desirable and would be preferred by many patients. On the other hand, there have been many attempts to prepare several drugs in the topical preparation for treatment of onychomycosis, for example, griseofulvin, but the results obtained were not very convincing. This could be the result of the scarce penetration of the drug into the deepest layers of nails. An appropriate and highly efficient vehicle for topical use in infected nails was found for the treatment of onychomycosis. These formulations should have the following characteristics because the penetration of the active ingredient is a slow process.

The drug should be applied in such a way that it can be spread freely in the fine tissue of the nail. It should be comfortable for the patient, easy to apply, it should not be applied very frequently, and it should be well tolerated.

Mycoses vary extensively in severity and can present as a superficial, subcutaneous, and/or systemic infection. Nowadays, there are efficient treatments for most superficial mycoses; there is still a need for new agents with convenient dosages and a low level of adverse effects for reducing serious subcutaneous and systemic fungal infections. In vitro Terbinafine shows broad spectrum activity against pathogenic fungi that are responsible for deep mycoses. There is no abundant clinical data for which it is suggested that Terbinafine's in vitro activity is reflected in its in vivo efficacy. (Hay R J, Br. J. Dermatol., November 1999; 141 Suppl. 56:36 - 40).

Data reported until the moment demonstrate that Terbinafine is a first-line treatment, useful for patients who have chromoblastomycosis as well as pulmonary aspergillosis. There is data that suggests Terbinafine being effective for histoplasmosis, treating fungal mycetoma, and cutaneous leishmaniasis. On the other hand, there is certain evidence that terbinafine has an activity synergic to amphotericin B, itraconazole, and fluconazole against clinical isolates of Candida species. That is how the therapeutic potential of Terbinafine is extended further than its actual use in acute and chronic dermatophytoses. Among allylaminal fungicides, Terbinafine is currently the most efficient one. In vitro, Terbinafine is highly active against a broad spectrum of pathogenic fungi. Clinical studies have demonstrated that terbinafine is efficient in the treatment of cutaneous and lymphocutanous sporotrichosis, and in several patterns of disseminated aspergillosis. Patients with chromoblastomycosis and other mycoses (phaeohyphomycosis, maduromycosis, and mucormycosis) have been treated. Old World cutaneous leishmaniasis, a parasitic sickness, has been treated with Terbinafine. These results suggest that the therapeutic potential of Terbinafine extends further than the current uses to be included in an array of subcutaneous and serious systemic and life-endangering mycoses. (Perez A, Mycoses, 1999, 42 (Suppl. 2):111-114).

### DETAILED DESCRIPTION OF THE INVENTION

Terbinafine's base is synthesized from N-methyl-1-1-naphtalenemethylamine base or hydrochlored with E-1.3 dichloropropane, the reaction is triggered by acetone boiled with 10% Nal and K₂CO₃ as a base. The intermediate resulting product E-N-3(3-chlorine-2propenyl)-N-methyl)-1-naphtalenemethylamine from the reaction sample, but this reacts with 3.3-dimethyl-1-butane under the presence of the catalytic Pd²⁺ (bis chlorinated (Benzonitrile) palladium(II)). CuI and piperidine base at room temperature (not less than 0.5% mol of Pd 2+ and 1% mol of CuI).

Preparation of Terbinafine Hydrochloride:

Terbinafine Hydrochloride is prepared by precipitation of a Terbinafine base dissolved in 2 propane or 1-I saturated, equivalent to dry 1-HCl. Afterwards, n-heptane is added. The final product is obtained.

The convenient polymers that form a film, which are not soluble in water are those preferred for formulation, like the one available under the registered brand GANTREZ ES. Poly (methyl vinyl ether-alt-maleic acid monobutyl ester) in 50% ethyl alcohol solution, base for the formation of an adhesive and cosmetic film. It forms a good, clear film that generates good adhesion and good resistance to humidity.

Likewise, it is known that ethyl alcohol is a product obtained from fractional distillation and in that formulation it is used as an antimicrobial preservative and solvent. Because it is well known that the concentrations used as an antimicrobial preservative are greater than or equal to 10% and as a solvent for films its concentration is variable.

On the other hand, Butylated Hydroxytoluene is used as an excellent antioxidant. In topical formulations, the range of allowed concentration is from 0.0075 to 0.1.

One of the objectives of this invention is to make a medication with a specific therapeutic action against fungi infection in fingernails and toenails possible.

To have a product that guarantees a broad spectrum of activity against most species of pathogenic fungi that are involved in fingernails and toenails, and further having a high level of activity against dermatophytes.

To have a medication whose therapeutic actions are not affected by the size of inoculation, besides it not having the potential of sensitization, which provides an excellent safety profile for the product for use in patients.

Another objective is to confer active compounds a quick and more elevated absorption.

### EXAMPLES

### Example 1

### Solubility at Room Temperature

TABLE 1
Tests of solubility for the active ingredient using mixing at 80 rpms at
room termperature.

| | | Terbinafine |
|---|---|---|
| Condition | Solvent | Hydrochloride |
| Constant mixing at room | Isopropyl Alcohol | +++ |
| temperature | Poly (methyl vinyl ether alt | +++ |
| | maleic acid monobutyl ester) | |
| | Ethyl Alcohol | ++++ |
| | Methanol | ++++ |
| | Water | ++ |

(+ = insoluble; ++ = slightly soluble; +++ = soluble; ++++ = very soluble)

From the solubility tests reported in Table 1, it is established that the most adequate solvent for dissolving Terbinafine Hydrochloride is Ethyl Alcohol at room temperature because it dissolves the active ingredient quickly without the need of applying heat.

In regards to the determination of the base for the varnish, the following components were approved: ethyl alcohol whose functions are that of being a solvent and a preservative, BHT as an antioxidant, poly (methyl vinyl ether alt maleic acid monobutyl ester) in 50% ethyl alcohol solution. This ingredient is used to form film active Terbinafine Hydrochloride.

Comparing the solubility of Terbinafine Hydrochloride and the Terbinafine base, it has been decided to use the hydrochloride form because solubility is greater in ethyl alcohol at room temperature with respect to the regular form.

The process is done in the following manner: all the process is done at room temperature, and for no reason should it be put to heat. In a bowl of the adequate size, Ethyl Alcohol is placed and Butylated Hydroxytoluene is added. The bowl is shaken until BHT is perfectly dissolved in the solvent, and then Terbinafine Hydrochloride is added and mixed until the solution is free of particles. Once it is completely incorporated, poly (methyl vinyl ether alt maleic acid monobutyl ester) in 50% ethyl alcohol solution is added. It is mixed until there is total homogeneity of the solution. The appearance of the varnish is yellow in color, transparent, and free of strange particles, with a nail polish smell.

Once the product was made, 3 samples were analyzed, taken from different parts of the bowl that contained the varnish solution of Terbinafine Hydrochloride. The analysis of its organoleptic, viscosity, density, and activity valuation characteristics were done in accordance with the analytic technique developed by Laboratorios Dermatológicos Darier.

The results are reported in Table 2.

TABLE 2
Results obtained in the valuation of the active
ingredients of the formula.

| | | Valuation | |
|---|---|---|---|
| Active Ingredient | | Results (%) | Specification |
| Terbinafine Hydrochloride | Sample 1 | 99.98 | 90.0 to 110.0% |
| | Sample 2 | 99.90 | |
| | Sample 3 | 100.02 | |

The previous results indicate that the product is homogenized due to uniformity of the results obtained, and each one of the excipients are compatible with the active ingredient, because there is no interference with the activity of Terbinafine Hydrochloride.

This invention presents 4 different formulas with different Terbinafine Hydrochloride concentrations using the aforementioned excipients.

Antimycotic Varnish 1%

| | PERCENTAGES |
|---|---|
| INGREDIENTS | (%) |
| Terbinafine Hydrochloride | 1.000 |
| BHT | 0.045 |
| Ethyl Alcohol | 25.005 |
| Poly (methyl vinyl ether alt maleic acid | 73.950 |
| monobutyl ester) in a 50% ethanol | |
| solution. | |

Antimycotic Varnish 2.5%

| | PERCENTAGES |
|---|---|
| INGREDIENTS | (%) |
| Terbinafine Hydrochloride | 2.500 |
| BHT | 0.045 |
| Ethyl Alcohol | 24.500 |
| Poly (methyl vinyl ether alt maleic acid | 72.955 |
| monobutyl ester) in a 50% ethanol | |
| solution. | |

Antimycotic Varnish 5.0%

| | PERCENTAGES |
|---|---|
| INGREDIENTS | (%) |
| Terbinafine Hydrochloride | 5.000 |
| BHT | 0.045 |
| Ethyl Alcohol | 24.005 |
| Poly (methyl vinyl ether alt maleic acid | 70.950 |
| monobutyl ester) in a 50% ethanol | |
| solution. | |

Antimycotic Varnish 10.0%

| | PERCENTAGES |
|---|---|
| INGREDIENTS | (%) |
| Terbinafine Hydrochloride | 10.000 |
| BHT | 0.045 |
| Ethyl Alcohol | 24.484 |
| Poly (methyl vinyl ether alt maleic acid | 65.471 |
| monobutyl ester) in a 50% ethanol | |
| solution. | |

| Result of the Valuation to the active | |
|---|---|
| ingredient for each one of the formulas. | Percentage % |
| Antimycotic Varnish 1% | 99.99% |
| Antimycotic Varnish 2.5% | 100.04% |
| Antimycotic Varnish 5% | 100.01% |
| Antimycotic Varnish 10% | 99.98% |

Hence, we can determine that the excipients are compatible with the different concentrations of Terbinafine Hydrochloride.

The varnishes at the different concentrations mentioned, 1%, 2.5%, 5%, and 10% in the finished product present minimal inhibitory concentrations similar to those specified in the pure standard. These results confirm that the formulas have no evident inhibitory effect in the activity of Terbinafine, according to Franz's studies. In preliminary experiments, the nails exposed to 10% Terbinafine Varnish inhibited fungal growth (Aditya Gupta MD, PhD, FRCPC).

What is claimed is:

1. A preparation for the care of fingernails and toenails comprising Terbinafine Hydrochloride, ethyl alcohol as a solvent or any of its derivatives or a combination that includes alkanes, BHT as an antioxidant, and poly (methyl vinyl ether alt maleic acid monobutyl ester) in 50% ethanol solution or any combinations thereof or including it as a former of film.

2. A preparation for the care of nails comprising a composition having Terbinafine Hydrochloride present at about 1.0 weight percent (wt %) to about 10.0 wt % based on the total weight of the composition, ethyl alcohol present at about 15.0 wt % to about 40 wt % based on the total weight of the composition, Butylated Hydroxytoluene (BHT) present at about 0.0075 wt % to about 0.1 wt % of the total weight of the composition, and poly (methyl vinyl ether alt maleic acid monobutyl ester) in 50% ethyl alcohol solution present at about 20 wt % to about 75 wt % based on the total weight of the composition.

3. A topical nail varnish comprising Terbinafine, a solvent, an antioxidant, and a polymeric film-former.

4. The topical nail varnish of claim 3, wherein said Terbinafine is an acid addition salt of Terbinafine.

5. The topical nail varnish of claim 4, wherein said acid addition salt of Terbinafine is Terbinafine Hydrochloride.

6. The topical nail varnish of claim 3, wherein said Terbinafine is present at about 1.0 wt % to about 10.0 wt % based on the total weight of the topical nail varnish.

7. The topical nail varnish of claim 3, wherein said solvent is ethyl alcohol.

8. The topical nail varnish of claim 7, wherein said ethyl alcohol is present at about 15.0 wt % to about 40 wt % based on the total weight of the topical nail varnish.

9. The topical nail varnish of claim 3, wherein said antioxidant is Butylated Hydroxytoluene (BHT).

10. The topical nail varnish of claim 9, wherein said BHT is present at about 0.0075 wt % to about 0.1 wt % based on the total weight of the topical nail varnish.

11. The topical nail varnish of claim 3, wherein said polymeric film-former is poly (methyl vinyl ether alt maleic acid monobutyl ester) in 50% ethyl alcohol solution.

12. The topical nail varnish of claim 11, wherein said poly (methyl vinyl ether alt maleic acid monobutyl ester) in 50% ethyl alcohol solution is present at about 20 wt % to about 75 wt % based on the total weight of the topical nail varnish.
